(19) United States
(12) Patent Application Publication
(10) Pub. No.: US 2009/0181094 A1
(43) Pub. Date: Jul. 16, 2009

(54) Molecular Cage for Sustained Release Control of Pharmaceutical and Cosmetic Agents

(76) Inventor: Eric Yueh-Lang Sheu, Lafayette, CA (US)

Correspondence Address: Eric Yueh-Lang Sheu, 7 Olde Creek Place, Lafayette, CA, 94549, US

(21) Appl. No.: 12/353,972

(22) Filed: Jan. 15, 2009

Related U.S. Application Data
(60) Provisional application No. 61/021,331, filed on Jan. 15, 2008.

(51) Int. Cl. A61K 9/00; A61P 43/00

(52) U.S. Cl. 424/486

(57) ABSTRACT

A method of making and using molecular cages is provided to control the release of molecules entrapped or caged within a caging molecule. The caging molecule may be a polymer, cellulose, or an organic and inorganic molecule that exhibits structural swelling, phase transitions, or structural rearrangement by changing thermodynamic parameters such as temperature and/or pressure. The caging molecule may also be accompanied by a co-caging molecule, which is also confined within the caging molecule, to further control the release of the caged molecules.

### CROSS-REFERENCE

This non-provisional application claims the benefit of provisional application No. 61/021,331 filed Jan. 15, 2008.

### BACKGROUND OF THE INVENTION

The invention relates to a method of making and using a dynamic molecular cage to confine a second molecule such as a pharmaceutical or cosmetic agent in order to control the rate of release of the second molecule to the environment external to the molecular cage. It also relates to a method of creating a molecular cage to confine or entrap the second molecule. That is, it is related to using solvents, temperature, pressure, or a combination thereof to initiate structure swelling, rearrangement, phase transition, or a combination thereof to cage the second molecule for controlling its release.

U.S. Pat. No. 4,869,904 describes using hydrophobicity of cyclodextrin to enhance drug solubility and bioavailability. U.S. Pat. No. 6,861,066, describes the concept of using modified C-60 fullerenes (“Buckyballs”) to control the release of entrapped molecules. A recent application, US Application No. 2006/0127430A1, describes a method of using zeolite to control the release of pharmaceutical and cosmetic active agents. In the above described references, the controlled release of the entrapped molecules depends on the natural cage structure of the molecules. The processes of making the cage-agent complex provided in those references are directed to introducing the agents into pre-existing caged structures. In contrast, the present system has a caging molecule that dynamically entraps or confines a second molecule. The present system does not include using dentrimers (star polymers) or molecules with a cage structure as their natural form such as cyclodextrins, modified C-60 fullerenes, or zeolites.

A cage structure can be formed by more than one molecule; examples include micelles, emulsion and microemulsion droplets, and liposomes. A system where one agent is caged by more than one molecule is referred to as a matrix, as disclosed in, for example, U.S. Pat. No. 5,334.392. The present system is also distinguished from these multi-component structures because a caging molecule of the present system is used to entrap or confine one or more second molecules.

### SUMMARY OF THE INVENTION

Many large molecules can assume different structures, depending on, for example, their orientation of polarity or exposure of hydrophilic-lipophilic moieties to the environment. Molecular structural formation and stability of dynamic systems are controlled in one part by thermodynamic forces, and therefore, thermodynamic parameters such as temperature or pressure may be varied to change the structures of micelle, emulsion, and liposomes, which are multi-component structural systems. The present systems described herein are single molecular systems where each entraps or confines one or more second molecules (caged molecules).

The present system includes molecules that form dynamic intra-molecular cages through specific control parameters, such as solubility, temperature, pressure, phase transition, supercriticality of fluids or a combination thereof.

A method of using a caging molecule to cage one or more second molecules such as a pharmaceutically active ingredient with therapeutic significance is also disclosed. In one example, the method involves using solvents or mixed solvents to swell or open up the caging molecule and then associating physically the second molecule to the caging molecule by, for example, adsoption, such that the second molecule partially loses its mobility. Then in another step such as evaporating the solvent, the caging molecule shrinks to form a tighter cage and the mobility of the second molecule is further reduced.

In another example, the swelling or opening of the caging molecules can be controlled also by temperature or by using a co-caging molecule for adjusting the degree of swelling and/or opening of the caging molecules through its affinity to the caging molecule and/or the caged molecules. The co-caging molecule gives some integrity to the caging molecule by sharing the caging space with the second molecule. One caging molecule may cage more than one caged molecule.

The second molecules may be released from their cages by thermal vibrations of the second molecules and/or the caging molecules. The probability of their vibration may, therefore, define the release rate of the second molecules from their caging molecules. The rate of release may also be moderated by the digestion of the caging molecule or by diffusion.

Mechanisms to promote or suppress their release rate include solvent extraction, and temperature, or pressure of extraction molecules. The release rate of the caged molecules within the caging molecules may be controlled for sustained release applications and/or protected from being extracted by certain solvents for abuse deterrence applications. The molecular caging system described herein may be used for sustained release control of topical cosmetic agents, therapeutic agents for topical uses, oral uses, or parenteral uses.

The molecular caging complex—the molecular entity including a caging molecule with caged molecules—can be in solid, semi-solid, liquid crystal, or liquid forms.

### DETAILED DESCRIPTION OF THE DRAWINGS

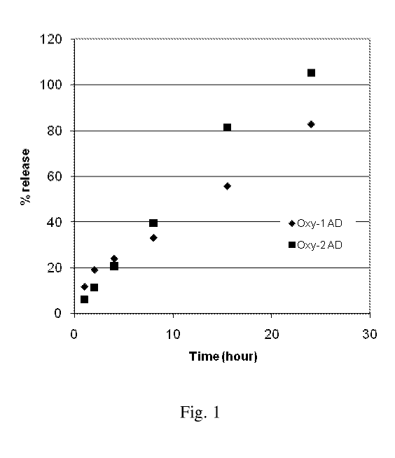

FIG. 1 shows a plot of an embodiment of the present invention.

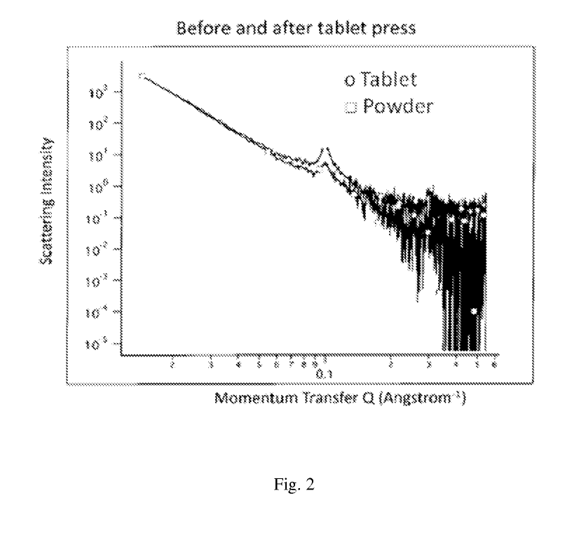

FIG. 2 shows another plot of an embodiment of the present invention.

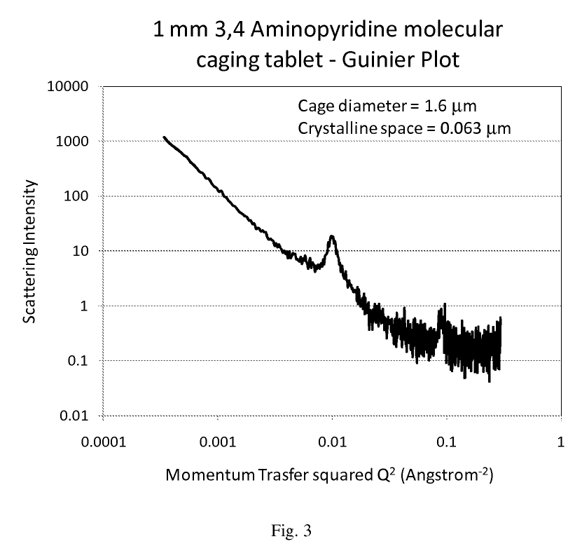

FIG. 3 shows still another plot of an embodiment of the present invention.

### DETAILED DESCRIPTION OF THE INVENTION

A method of making a sustained release formulation is provided. Molecules that can be used for caging purposes include those that exhibit various dynamic structural forms, particularly those that can open (e.g. swell or extend) or close (e.g. shrink or contract) upon, for example, evaporating the solvent, adding other solvents, heating and cooling, or pressurizing and de-pressurizing the solvent with the formulation.

In polymer systems, there often exists a so-called theta-point where the polymer is in its most extended structure and shrinks when the thermal dynamic parameters move away from this point. By a similar mechanism, a caging molecule can be swelled or “extended” by any physical or chemical means, or a combination thereof to allow the second molecule to be caged move into the proper position so that when the parameters resulting in the swelling or extension of the caging molecule are changed, the caging molecule shrinks to cage the second molecule.

Caging molecules other than polymers may be polysaccharides, biomolecules, or inorganic molecules such as silica, to name a few, so long as they exhibit swellable or extendable structures at a thermodynamic condition and can cage molecules when the thermodynamic condition is properly varied, similar to the way polymeric structures change upon varying the temperature, solvent quality, and/or pressure.

A caging molecule includes but not limited to: ethylene vinyl acetate, polyvinypirollidone, polyvinyl acetate, ethyl cellulose, polyethylene glycol, polypropylene glycol, polyoxyethylene sorbitan fatty acid esters, polysorbates, sobitan esters, chitosan, guar gum, gelatine, methyl cellulose, hydroxypropyl methyl cellulose, cellulose acetate phthalate, hydroxypropyl ethyl cellulose, polycarprolactone, poly(urethanes), poly(siloxanes), poly(methyl methacrylate), poly(vinyl alcohol), poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, polyethylene oxide (PEO), polypropylene oxide (PPO), PEO and PPO block co-polymers or tri-block co-polymers.

During the above described caging processes, additives may be incorporated to control the degree of swelling or opening of the caging molecules, which may also lead to controlling the release rate of the caged, second molecules.

A co-caging molecule may be added to further control the caging efficiency. For example, a co-caging molecule that can be physically adsorbed to a second molecule to be caged may exhibit a higher affinity to the caging molecule than the second molecule, and therefore, under this condition when the co-caging molecule together with the second molecule to be caged is introduced to the caging molecule that is swelled in a solvent, the co-caging molecule and the second molecule may form a complex with the caging molecule. The co-caging molecule would then facilitate the second molecule to be associated with the caging molecule. When the solvent is evaporated, the co-caging molecule and the second molecule become caged within the caging molecule. The caging process can be controlled by a solvent-non-solvent addition, solvent evaporation, heating-cooling cycle, pH adjustment, or pressurization-de-pressurization cycle.

A co-caging molecule includes but not limited to: beeswax, carnauba wax, emulsifiable wax, emulsifiers such as acacia, anionic emulsifying wax, calcium stearate, carbomers, cetostearyl alcohol, cetyl alcohol, cholesterol, diethanolamine, PEG, ethylene glycol palmitostearate, glycerin monostearate, hydroxypropyl cellulose, hypromellose, lanolin, lanolin alcohol, lecithin, medium chain triglycerides, methylcellulose, mineral oil, fatty acids, monobasic sodium phosphate, monoethanolamine, nonionic emulsifying wax, oleic acid, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate di-hydrate, sodium lauryl sulfate, sorbitan esters, stearic acid, sunflower oil, tragacanth, triethanolamine, xanthan gum; food oils, such as diglyceride, triglyceride, olive oil, canola oil, peanut oil.

Various different hydrophilic-hydrophobic combinations are possible with the caging molecule/co-caging molecule complex. For example, a caging molecule/co-caging molecule complex may be hydrophilic/hydrophobic (e.g. guar gum/wax), hydrophilic/hydrophilic (e.g. polyvinypirollidone/PEG), hydrophobic/hydrophobic (e.g. ethyl cellulose/wax), or hydrophobic/hydrophilic (e.g. ethyl cellulose/PEG). It is also possible that the caging molecule and/or co-caging molecule may be suitably amphiphilic.

The release rate of the caged, second molecules can depend on the thermal vibration of the second molecule and the thermal vibration or swelling of the caging molecule, or the affinity of the caged molecule to the environment outside the caging molecule. For example, when the caged molecules are in a solvent that dissolves the second molecule but not the caging molecule, the chemical potential creates a force from the solvent that pulls the caged molecule from the caging molecule. In this situation, the release rate would depend on the physical barrier set by the caging molecule and its structural vibrations. This feature enables the caging system to protect the second molecule from being rapidly extracted. Therefore, the present system is applicable for controlling the release rate of the second molecule as well as deterring the abuse of the second molecule such as narcotics.

A second molecule may be a pharmaceutical agent such as a therapeutic or diagnostic agent, or a cosmetic agent. A co-caging agent may also be provided so that the molecular caging molecule cages the pharmaceutical or cosmetic agent while the co-caging agent fills the rest of space of the cage. A co-caging molecules may be used to moderate hydrophobic/lipophilic, dielectric, and/or affinity differences between the caging molecule and the second molecule. A sustained release control of the caged complex is achieved not only by using the differences of affinity between the second molecule and the caging molecule to the environment at the site of administration but also by using the affinity of the co-caging material to the environment.

A second molecule includes but not limited to: local analgesic drugs such as cocaine, procaine, chloroprocaine, tetracaine, benzocaine, lidocaine, etidocaine, bupivacaine, andropivacine; general analgesic drugs such as diazepam, lorazepam, etomidate, fentanyl, morphine, halothane, isoflurane, ketamine, midazolam, propofol, sevoflurane, and thiopental; anxiolytics and sedatives such as alprazolam, buspirone, chlordiazepoxide, clonazepam, clorazepate, diazepam, flumazenil, flurazepam, halazepam, lorazepam, midazolam, oxazepam, phenobarbitol, prazepam, temazepam, temazepam, thiopentyl, triazolam, zaleplon, and zolpidem; non-steroid anti-inflammatory drugs (NSAID) such as acetaminophen, acetylcysteine, aspirin, celecoxib, indomethacin, meloxicam, naproxen, phenylacetic acids, phenylbutazone, piroxicam, refocoxib, sulindac, and tolmetin; opioids such as buprenorphine, butorphanol, codeine, dextromethorphan, dextropropoxyphene, diphenoxylate, fentanyl, heroin, hydrocodone, hydromorphone, meperidine, methadone, morphine, nalbuphine, naloxone, naltrexone, oxycondone, oxymorphone, and pentazocine; steroids such as aminoglutethimide, cortisol, cosyntropin, desoxycorticosterone, dexamethasone, fludrocortisone, fluocinonide, hydrocortisone, ketoconalole, methylprednisolone, metyrapone, prednisone, spironolactone, and triamcinolone; biomolecule drugs such as interferon; monoclonal and antibodies; peptide drugs such as cacitonin, cyclosporine; and bioregulators such as plasmid DNA, RNA, siRNA, and human growth hormone.

An example of preparing a hydrophilic caging molecule with a hydrophilic co-caging molecule and a secondary molecule is as follows: Dissolve or disperse drugs, for example, a bupivacaine salt, in polyethylene glycol (PEG) in liquid form at room temperature for a low molecular weight PEG, such as PEG 200, PEG 300, or PEG 400 or melted form for a high molecular weight PEG, such as PEG 100. Add a caging molecule, such as hydroxypropyl methyl cellulose (HPMC) at an elevated temperature with some water to swell HPMC and mix to let HPMC cage or confine PEG and the drug. Maintain the elevated temperature until the water evaporates or apply vacuum if the drug cannot withstand the high temperature for an extended period of time, and the system becomes cake-like or powdery. At this point, the cage is formed with PEG and the drug in the cage. Cool gradually or rapidly depending on the PEG used and its compatibility with the LogP value of the drug (the value that describes the hydrophilicity of the drug—high LogP means the drug is more hydrophobic. The HPMC cage will form when drug-PEG affinity is stronger than drug-HPMC affinity. A drug LogP range of −5 to +6 is applicable, although not limited to that range.

An example of preparing a hydrophobic caging molecule with a hydrophilic co-caging molecule and a secondary molecule is as follows. Dissolve or disperse drugs, for example, a bupivacaine salt, in PEG in liquid form (at room temperature for a low molecular weight PEG, such as PEG 200, PEG 300, or PEG 400) or melted form for a high molecular weight PEG, such as PEG 100. Add polyacrylate solution (for example, in 3:2 volume ratio of acetone and methanol) to disperse the PEG/drug. Gradually evaporate the solvents to let polyacrylate cage or confine PEG and the drug. The polyacrylate cage would form with PEG and drug in the cage only when the drug-PEG affinity is higher than drug-polyacrylate, which makes the drug stay adhered to PEG during the solvent evaporation process.

An example of preparing a hydrophobic caging molecule with a hydrophobic co-caging molecule and a secondary molecule is as follows: Dissolve or disperse drugs, for example, a bupivacaine base, in fatty acid at room temperature or at an elevated temperature depending on the fatty acid melting point. For injectable dosage form, fatty acids chosen are liquid fatty acids at room temperature, and for oral dosage form, fatty acids chosen are solid at room temperature but becomes liquid at an elevated temperature. Add polyacrylate solution (for example, in 3:2 volume ratio of acetone and methanol) to disperse the PEG/drug. Gradually evaporate the solvents to let polyacrylate cage or confine the fatty acid and the drug. The polyacrylate cage would form with the fatty acid and the drug in the cage only when the drug-fatty affinity is higher than the drug-polyacrylate affinity (depending on the LogP value of the drug), which makes the drug stay adhered to the fatty acid during the solvent evaporation process.

An example of a formulation is a linear polymer such as polyvinylpyrollidone (PVP) as a caging molecule, an oil like material such as lipid, fatty acids, triglyceride, or oil as a co-caging molecule, and a pharmaceutical compound (“drug”) as a second molecule caged in the PVP cage. The co-caging material fills the cage to mediate between the drug and the caging molecule. When the formulation is administered, for example, to a subcutaneous environment comprising adipose cells, the largely hydrophobic environment of the subcutaneous space and the hydrophilic PVP repel each other to prevent the drug from being drawn out of the cage too rapidly. On the other hand, a subcutaneous space with an aqueous medium also cannot draw the drug out rapidly because of the presence of the hydrophobic oil as the co-caging material. As a result, the formulation is a caged complex that achieves a sustained release of the drug over an extended period of time.

For an example of an implant, a higher molecular weight material, such as chitosan can be used as a caging molecule, and a high melting point hydrophobic material, such as cholesterol or tri-glyceride, can be used as a co-caging material. The sustained release regulation of the second molecule caged within chitosan is achieved by the hydrophilic-hydrophobic zone formed by the molecular caged complex where chitosan forms the hydrophilic layer and oil-like triglyceride forms the hydrophobic zone.

For an example of an oral administration, guar gum as a caging molecule may serve as the hydrophilic layer and wax as a co-caging molecule may serve as the hydrophobic zone. Guar gum can be degraded in the colon by bacteria (flora) but it is soluble neither in alcohol nor wax. Therefore, it can deter abuse by largely preventing the encaged drug from being dissolved in alcohol and it can also sustain release because of the hydrophilic-hydrophobic zone formed by the complex. It can also be a colon delivery system because guar gum degrades in the colon. Other system, such as gelatin system can be used as the caging molecule and wax as the co-caging agent, in which the gelatin will gradually swell as water gradually enter the wax zone. The rate of gelatin swelling (being retarded by wax) will define the rate of the sustain release.

### EXAMPLES

The following examples illustrate the embodiments of the present invention. As examples they are not intended to limit the scope of the invention. All quantities are in weight %.

### Example 1

### A Method Using Molecular Caging Concept for Formulating Abuse Deterrence Drug Dosage Form Using Naltrexone as a Model Compound

Naltrexone is used as a model therapeutic agent for opioid, such as Oxycodone, which is known to be widely abused. Naltrexone, in this example as a second molecule, is caged in guar gum, a caging molecule in this example, with bee's wax and carnauba wax as co-caging agents. In the process, weighed naltrexone is dissolved in ethyl alcohol, the resulting solution is added to another container with bee's wax, carnauba wax, guar gum and sorbitan monooleate, and then the container is heated to 80° C. to melt and dissolve bee's wax and carnauba in the alcohol with dissolved naltrexone. Sorbtan monooleate acts as an emulsifier to increase miscibility of naltrexone to wax. Guar gum, which does not dissolve in either alcohol or melted wax, is swelled at the high temperature. In the meantime, the dielectric constant of ethyl alcohol decreases as reported by Crain (C. M. Crain, “The dielectric constant of several gases at a wave-length of 3.2 centimeters,” Physical review, Vol. 74, No. 6, 1948) and the alcohol becomes more hydrophobic, thus prompting neltrexone to associate with the wax while sorbitan monooleate acts to ensure miscibility. Under the thermodynamic equilibrium, one naltrexone/wax complex was associated with one heat extended guar gum molecule because guar gum molecules were provided in excess of the naltrexone/wax complexes. The container was then opened while the temperature was maintained at 80° C. Ethyl alcohol gradually evaporated, and the naltrexone/wax complexes were caged in the guar gum molecules with waxes serving as the co-caging agent to restrict rapid swelling of guar gum in aqueous media. The resulting malleable putty-like formulation was filled into hard gelatin capsules. Table 1 lists the compositions. A simulated abuse protocol using 80 proof alcohols to extract naltexone was tested. Table 2 shows the results.

TABLE 1
Formulation I - compositions

| Ingredient | Weight (mg) |
|---|---|
| Naltrexone | 190.9 |
| Guar gum | 635.8 |
| Bee wax | 220.3 |
| Carnauba wax | 270.4 |
| Sorbitan monoooleate | 152.6 |

TABLE 2
Extraction of naltrexone from formulation I at room temperature

| Time of | Extraction (w/w %) in various media | | |
|---|---|---|---|
| extraction | Orange Juice | PH = 9 buffer | 80 Proof Alcohol |
| T = 0 | ND | ND | ND |
| 5 minutes | 0.03 | 0.03 | 0.05 |
| 1 hour | 0.2 | 0.1 | 0.4 |
| 2 hours | 0.5 | 0.2 | 0.6 |
| Overnight | 4.4 | 1.2 | 3.3 |

ND = non-detectable

### Example 2

Preparation of formulation II using Table 3 compositions. Dichloromethane (10 ml) ethyl alcohol (2 ml) were added to Part A comprising naltrexone, guar gun, and cellulose acetate phthalate (CAP) in a container. Cellulose acetate pthalate (CAP) and naltrexone were fully dissolved and guar gum was suspended and swelled by tumbling the container. The container was heated to 65° C. and maintained at 65° C. until all the solvents evaporated. Part B containing carnauba wax and sugar ester 190 were heated to 70° C. and mixed well. Then Part A and Part B were mixed, and the result was filled into hard gelatin capsules.

TABLE 3
Formulation II - compositions

| Ingredient | Weight (mg) |
|---|---|
| Part A | |
| Naltrexone | 494.6 |
| Guar gum | 3022 |
| Cellulose acetate pthalate (CAP) | 1005 |
| Part B | |
| Carnauba wax | 2526 |
| Sugar ester 190 | 3020 |

### Example 3

Preparation of formulation III using Table 4 compositions. Naltrexone and bee's wax were dissolved in dichloromethane in container A; guar gum was added to container A and mixed well at 70° C. In a separate container (container B) 2 ml of water was added to a gelatin, and container B was heated to 70° C. and mixed until homogeneous. Container A and B were then mixed, dichloromethane was evaporated, and the container was gradually cooled to room temperature. Table 5 gives the results of the abuse deterrence tests.

TABLE 4
Formulation III - compositions

| Ingredient | Weight (mg) |
|---|---|
| Naltrexone | 62.7 |
| Guar gum | 400 |
| Bee wax | 130.3 |
| Gelatin | 599.7 |

TABLE 5
Abuse deterrence test for Formulation II and III in 80 proof alcohol.

| | |
|---|---|
| | Extraction (w/w %) |
| Time of | in 80 proof alcohol |

| extraction | Formulation II | Formulation III |
|---|---|---|
| T = 0 | 0.1 | 0.02 |
| 5 minutes | 0.2 | 0.1 |
| 1 hour | 0.6 | 0.4 |
| 2 hours | 0.7 | 0.8 |
| Overnight | 2.6 | 3.4 |

### Example 4

10 gm batch OXY-1 formulation compositions
Part A

| | |
|---|---|
| Naltrexone (wt %) | 5 |
| Cellulose Acetate | 10 |
| Pthalate (wt %) | |
| Guar Gum (wt %) | 30 |

Part B

| | |
|---|---|
| Carnauba wax (wt %) | 25 |
| Sugar ester 190 (wt %) | 30 |

Process:
1. Add 10 ml of dichloromethane to part A
2. Add 2 ml of ethanol to part A and mix well
3. Heat to 65° C. to slowly evaporate the solvents
4. Heat part B to 70° C. and mix well, then mix with part A and continue mixing
5. Load the mixture into syringe
6. Load the desired amount from the syringe to a gelatin capsule
7. Theoretical Naltrexone weight=5%

### Example 5

6.6 gm batch OXY-2 capsule formulation composition
Part A

| | |
|---|---|
| Naltrexone (wt %) | 8 |
| Bee Wax (wt %) | 15.5 |
| Sugar ester 190 (wt %) | 16.5 |

Part B

| | |
|---|---|
| Guar Gum (wt %) | 60 |

### Dissolution and Results of Examples 4 and 5

1. Formulations were encapsulated in 0-size gelatin capsules
2. Capsules were situated in a USP apparatus 1 basket, then submerged in a glass jar with media
3. Media=de-ionized water
4. Temperature=37.0° C.
5. Instrument: orbital shaker at 100 rpm
6. Oxy-1 and Oxy-3 results are shown below.

### Example 6

Formulation Composition

| | |
|---|---|
| 3,4-Diaminopyridine (wt %) | 12.65 |
| Carnauba wax (%) | 11.42 |
| Pearlitol 50C (%) | 63.53 |
| Compritol 888 ATO (wt %) | 9.63 |

Process:
1. Mix 3,4 diaminopyridine, carnuba wax and Compritol powders in a glass gar and heat to 75° C. until both carnauba wax and Compritol are melt and all the ingredients well mixed
2. Cool the mixture down to room temperature
3. Blending (2) into powder
4. Roller compact the mixture
5. Sieve through mesh=30 screen into size 30 powder
6. Tablet into 1 mm thickness round tablets

Small Angle X-ray Scattering:

FIG. 2 shows small angle X-ray scattering data of the example 6 3,4 diaminopyridine tablets and the roller compacted powder used for tablet press. Both spectra in FIG. 2 show a well defined peak at about 0.1 Å⁻¹ indicating a 0.063 μm structure, which comes from the Pearitol 50C, and the small angle region of the tablet spectrum shows a linear line when presented in the Guinier format (see FIG. 3). By Guinier analysis, the cage dimension was estimated to be about 1.6 μm.

What is claimed is:

1. A method of making a molecular caging complex as a sustained release formulation, comprising:
providing caging molecules and second molecules to be caged by the caging molecules, where caging molecules are provided in excess of the second molecules;
mixing the caging molecules and the second molecules;
opening the caging molecules to allow the second molecules to be associated with the caging molecules;
closing the caging molecules such that one or more second molecules are confined within one caging molecule to make a molecular caging complex,
wherein opening and closing the caging molecules are controlled by changing thermodynamic parameters including temperature and/or pressure, evaporating a solvent of the caging molecule, inducing a phase transition, or taking a solution with the caging molecules into a supercritical state, and
wherein the caging molecules are polymers, biomolecules, polysaccharides, or organic or inorganic molecules in which each forms a cage structure that confines one or more of the second molecules.

2. A method according to claim 1, further comprising:
providing co-caging molecules in addition to the second molecules to incorporate within the caging molecules, thereby further controlling and facilitating the confinement of the second molecules within the caging molecules.

3. A method according to claim 1, further comprising:
providing co-caging molecules in addition to the caging molecules to incorporate within the caging molecules, thereby mediating between the caging molecules and the second molecules to accommodate for hydrophobic and lipophilic differences between the caging molecules and the second molecules.

4. A method according to claim 1, further comprising:
providing co-caging molecules in addition to the caging molecules to incorporate within the caging molecules, thereby mediating between the caging molecules and the second molecules to accommodate for dielectric differences between the caging molecules and the second molecules.

5. A method according to claim 1, further comprising:
providing co-caging molecules in addition to the caging molecules to incorporate within the caging molecules, thereby mediating between the caging molecules and the second molecules to accommodate for differences of affinity between the caging molecules and the second molecules to the environment external to the caging molecule.

6. A method according to claim 2, wherein the co-caging molecules are a hydrophobic material comprising: a wax, oil, lipid, fatty acids, cholesterol, or triglyceride.

7. A method according to claim 1, wherein the second molecules are a drug subject to sustained time release.

8. A composition comprising:
one or more second molecules;
a caging molecule for confining one or more second molecules within the caging molecule; and
a co-caging molecule to fill the caging molecule to further confine the second molecules; and the co-caging molecule also being confined within the caging molecule,
wherein the caging molecule is a polymer, biomolecule, polysaccharide, or organic or inorganic molecule in which each forms a cage structure that confines one or more of the second molecules.

9. A composition according to claim 8, wherein the caging molecule is hydrophilic and the co-caging molecule is hydrophobic.

10. A composition according to claim 8, wherein the second molecules are a drug subject to sustained release.

11. A composition according to claim 8, wherein the co-caging molecule is a hydrophobic material comprising: a wax, oil, lipid, fatty acids, cholesterol, or triglyceride.

12. A method of treatment, comprising:
administering to a subject a formulation containing a caging complex, wherein the caging complex comprises:
one or more second molecules;
a caging molecule for confining one or more second molecules within the caging molecule;
a co-caging molecule to fill the caging molecule to further confine the second molecules; and the co-caging molecule also being confined within the caging molecule,
wherein the caging molecule is a polymer, biomolecule, polysaccharide, or organic or inorganic molecule in which each forms a cage structure that confines one or more of the second molecules.

13. A method of treatment according to claim 12, wherein the caging complex is administered orally or parenterally.

14. A method of treatment according to claim 12, wherein the caging complex is administered by implanting the complex within the subject.

15. A method of treatment according to claim 12, wherein the caging molecule is hydrophilic and the co-caging molecule is hydrophobic.

16. A method of treatment according to claim 12, wherein the second molecules are a drug subject to sustained release.

17. A method of treatment according to claim 12, wherein the co-caging molecule is a hydrophobic material comprising: a wax, oil, lipid, fatty acids, cholesterol, or triglyceride.

18. A method of deterring drug abuse, comprising:
providing a formulation containing a caging complex, wherein the caging complex comprises:
one or more second molecules;
a caging molecule for confining one or more second molecules within the caging molecule;
a co-caging molecule to fill the caging molecule to further confine the second molecules; and the co-caging molecule also being confined within the caging molecule,
wherein the second molecules are prevented from being abused by having the caging molecule acting as a barrier against rapid release to the environment external to the caging complex, and
wherein the caging molecule is a polymer, biomolecule, polysaccharide, or organic or inorganic molecule in which each forms a cage structure that confines one or more of the second molecules.

19. A method according to claim 18, wherein the caging molecule is hydrophilic, hydrophobic, or amphiphilic and the co-caging molecule is hydrophilic, hydrophobic, or amphiphilic.

20. A method according to claim 18, wherein the second molecules are a drug subject to sustained release.

21. A method according to claim 18, wherein the co-caging molecule is a hydrophobic material comprising: a wax, oil, lipid, fatty acids, cholesterol, or triglyceride.
